(19) United States
(12) Patent Application Publication
(10) Pub. No.: US 2019/0281865 A1
(43) Pub. Date: Sep. 19, 2019

(54) Process and apparatus of producing beverge

(71) Applicants: Chang-Lu Hsu, Kaohsiung (TW); Yi-Jinn Chen, Kaohsiung (TW); Shui-Chen Huang, Kaohsiung (TW); Jung-Lung Lo, Taipei (TW); Kou-Sen Kou, Kaohsiung (TW)

(72) Inventors: Chang-Lu Hsu, Kaohsiung (TW); Yi-Jinn Chen, Kaohsiung (TW); Shui-Chen Huang, Kaohsiung (TW); Jung-Lung Lo, Taipei (TW); Kou-Sen Kou, Kaohsiung (TW)

(21) Appl. No.: 16/427,343

(22) Filed: May 31, 2019

(51) Int. Cl. A23L 2/38; A23L 2/04; A23L 2/72; B01D 33/11; B01D 33/46; B65B 3/04; B65B 55/10; A61K 36/33; A61K 31/15

(52) U.S. Cl. CPC A23L 2/382; A23L 2/04; A23L 2/72; B01D 33/11; B01D 33/466; A23Y 2300/55; B65B 55/10; A61K 36/33; A61K 31/15; A23V 2002/00; A23Y 2220/03; B65B 3/04

(57) ABSTRACT

A process of producing beverage includes the steps of washing cactus fruit and pitaya with clean water; drying the clean cactus fruit and pitaya; juicing the cactus fruit to obtain juice of the cactus fruit and juicing the pitaya to obtain juice of the pitaya respectively; filtering the juice of the cactus fruit and the juice of the pitaya respectively; mixing the filtered juice of the cactus fruit and the filtered juice of the pitaya with water to obtain a solution; pouring yeast and gram-positive bacteria in the solution wherein the yeast is Saccharomyces boulardii and the gram-positive bacteria includes Lactobacillus acidophilus and Bifidobacterium longum; and fermenting the solution until beverage is produced wherein fermentation temperature is between 26° C. and 35° C. and fermentation time lasts for 3 to 10 days.

### CROSS-REFERENCE TO RELATED APPLICATION

This application is a continuation-in-part (CIP) of U.S. patent application Ser. No. 15/808,881, filed Nov. 9, 2017, now abandoned, which is incorporated herein by reference in its entirety.

### BACKGROUND OF THE INVENTION

### 1. Field of the Invention

The invention relates to process of producing beverage by fermenting and more particularly to a process of producing beverage by fermenting cactus fruit and pitaya (i.e., dragon fruit) and an apparatus thereof.

### 2. Description of Related Art

A conventional process of producing cactus and vegetable based enzyme comprises cutting cactus and vegetable, selecting 1×10⁵-9×10⁵ CFU/ml of at least one of yeast, acetic acid, lactic acid, and fermentation starter as species, mixing the cut cactus and vegetable with the species to obtain a mixture, and adding sugar and the mixture to a container for fermentation for 150 to 200 days.

Another conventional process of producing wine by fermenting pitaya and Siraitia grosvenorii comprises washing and cutting peeled pitaya, corn and Siraitia grosvenorii to obtain a mixture, and adding yeast, water, sugar, and acid to the mixture for fermentation 3 to 5 months, thereby obtaining a first solution; mixing cut corn with water and sugar, and adding yeast to the mixture for fermentation 3 to 5 months, thereby obtaining a second solution; and boiling the first and second solutions and sealing same to produce wine.

However, these processes are complicated, nutrients in the finished product are lost, and the production time is excessively long. Thus, the need for improvement still exists.

### SUMMARY OF THE INVENTION

It is therefore one object of the invention to provide a process of producing beverage comprising the steps of washing cactus fruit and pitaya with clean water; drying the clean cactus fruit and pitaya; juicing the cactus fruit to obtain juice of the cactus fruit and juicing the pitaya to obtain juice of the pitaya respectively; filtering the juice of the cactus fruit and the juice of the pitaya respectively; mixing the filtered juice of the cactus fruit and the filtered juice of the pitaya with water to obtain a solution; pouring yeast and gram-positive bacteria in the solution wherein the yeast is Saccharomyces boulardii and the gram-positive bacteria includes Lactobacillus acidophilus and Bifidobacterium longum; and fermenting the solution until beverage is produced wherein fermentation temperature is between 26° C. and 35° C. and fermentation time lasts for 3 to 10 days.

The above and other objects, features and advantages of the invention will become apparent from the following detailed description taken with the accompanying drawings.

### BRIEF DESCRIPTION OF THE DRAWINGS

FIG. 1 is a flow chart illustrating a process of producing beverage by fermenting cactus fruit and pitaya according to the invention;

FIG. 2 is a perspective view of a drying device of an apparatus of producing beverage by fermenting cactus fruit and pitaya according to the invention;

FIG. 3 is a side elevation, in part section of a juicing device of the apparatus;

FIG. 4 is a longitudinal sectional view of a filtering device of the apparatus;

FIG. 5 is a perspective view of a fermenting device of the apparatus;

FIG. 6 schematically depicts a sterilizing device of the apparatus;

FIG. 7 is a sectional view taken along line A-A of FIG. 6;

FIG. 8 is a chart of days versus values for anti free-radical DPPH activity before and after fermentation; and

FIG. 9 is a bar chart of days versus values for anti free-radical before and after fermentation.

### DETAILED DESCRIPTION OF THE INVENTION

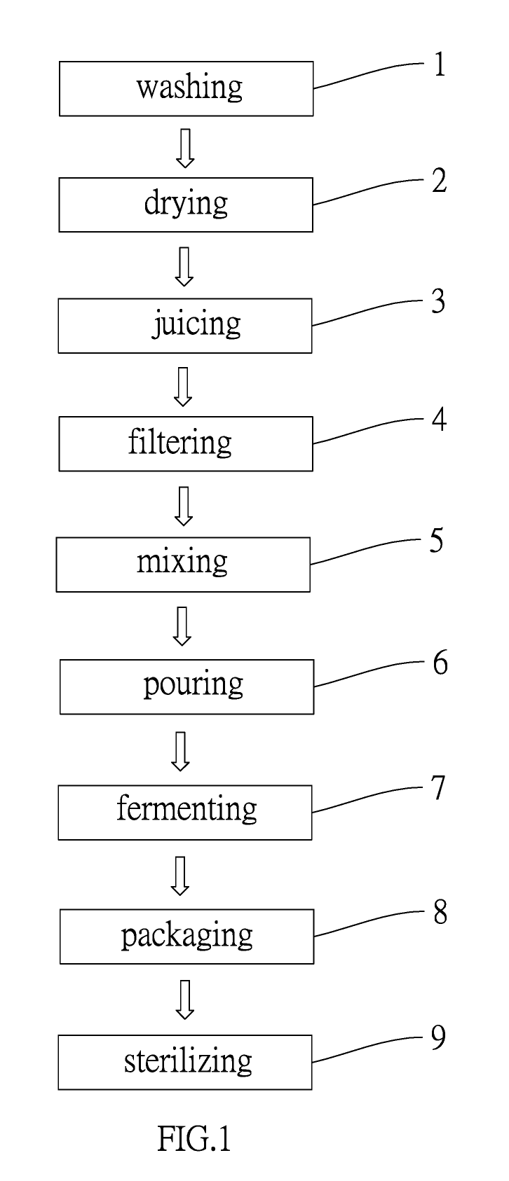

Referring to FIG. 1, a process of producing beverage by fermenting cactus fruit and pitaya in accordance with the invention illustrated. The process comprises the following steps:

Step 1: Washing cactus fruit and pitaya with clean water.

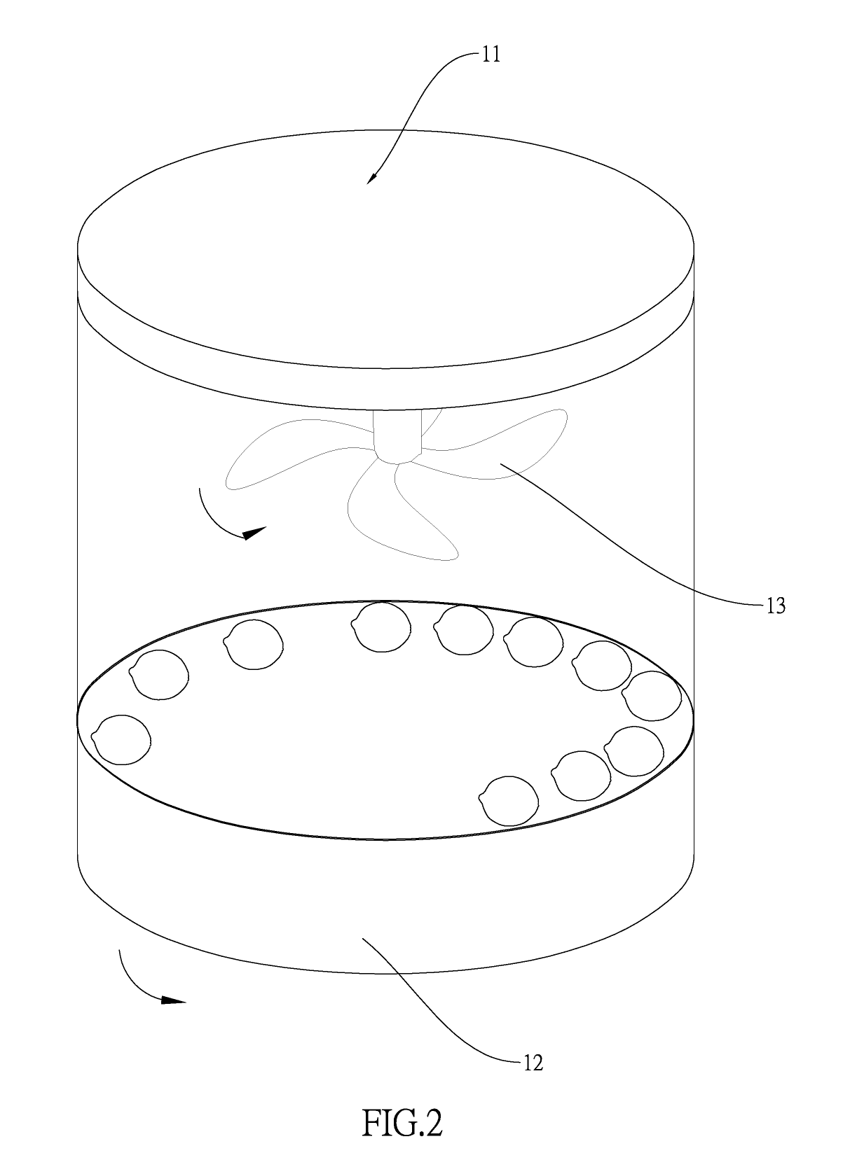

Step 2: Drying the clean cactus fruit and pitaya in a drying device 11 (FIG. 2) to remove water out of peels of both cactus fruit and pitaya.

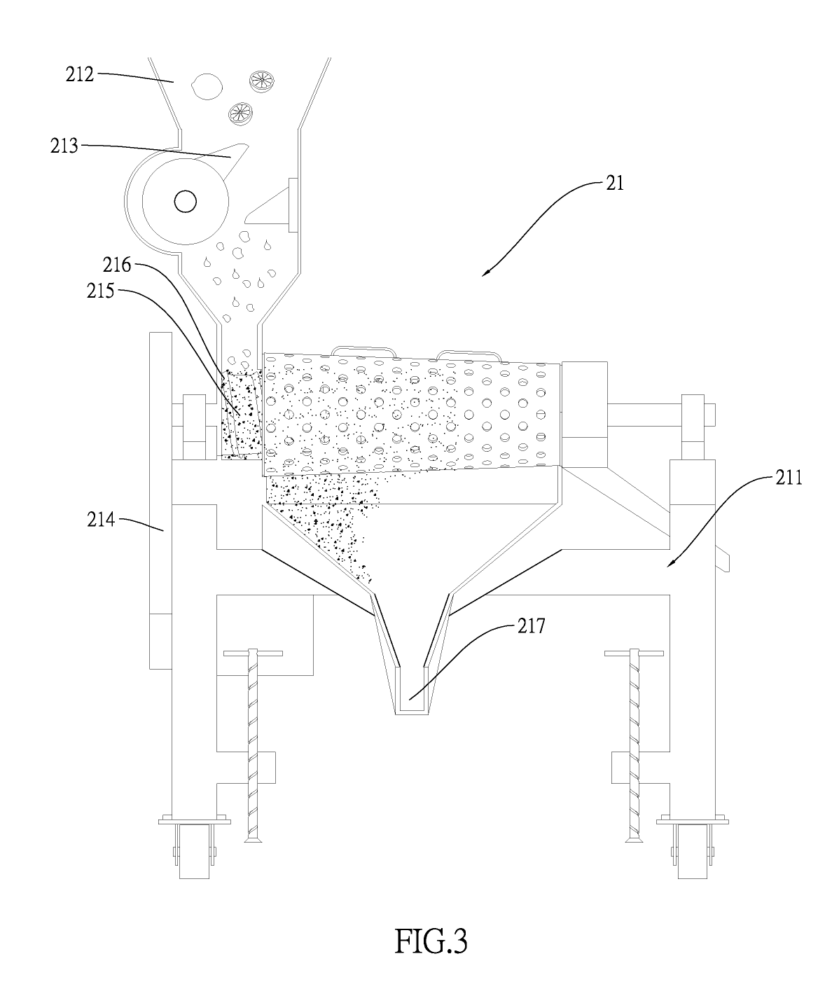

Step 3: Juicing the cactus fruit in a juicing device 21 (FIG. 3) to obtain juice of the cactus fruit and juicing the pitaya in the juicing device 21 (FIG. 3) to obtain juice of the pitaya respectively. It is noted that peels of both cactus fruit and pitaya are juiced for increasing nutrition of the juices.

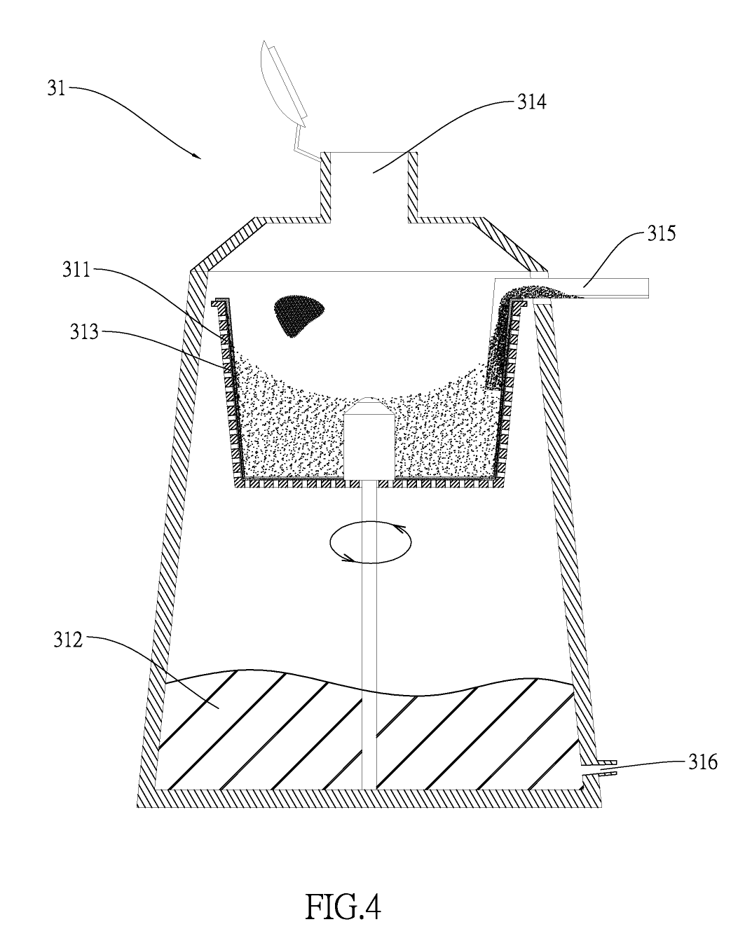

Step 4: Filtering the juice of the cactus fruit and the juice of the pitaya in a filtering device 31 (FIG. 4) respectively so as to obtain pure juice of the cactus fruit and pure juice of the pitaya respectively.

Step 5: Mixing the pure juice of the cactus fruit and the pure juice of the pitaya with water to obtain a solution wherein a volumetric ratio of the filtered juice of the cactus fruit, the filtered juice of the pitaya, and water is 0.05-0.1:1-5:1-5.

Step 6: Pouring yeast and gram-positive bacteria in the solution wherein the yeast is Saccharomyces boulardii, the gram-positive bacteria includes Lactobacillus acidophilus and Bifidobacterium longum, and a volumetric ratio of Saccharomyces boulardii, Lactobacillus acidophilus, and Bifidobacterium longum is 1-5:1-3:1-3 and a volumetric ratio of the solution and both yeast and gram-positive bacteria is 97-99:1-3.

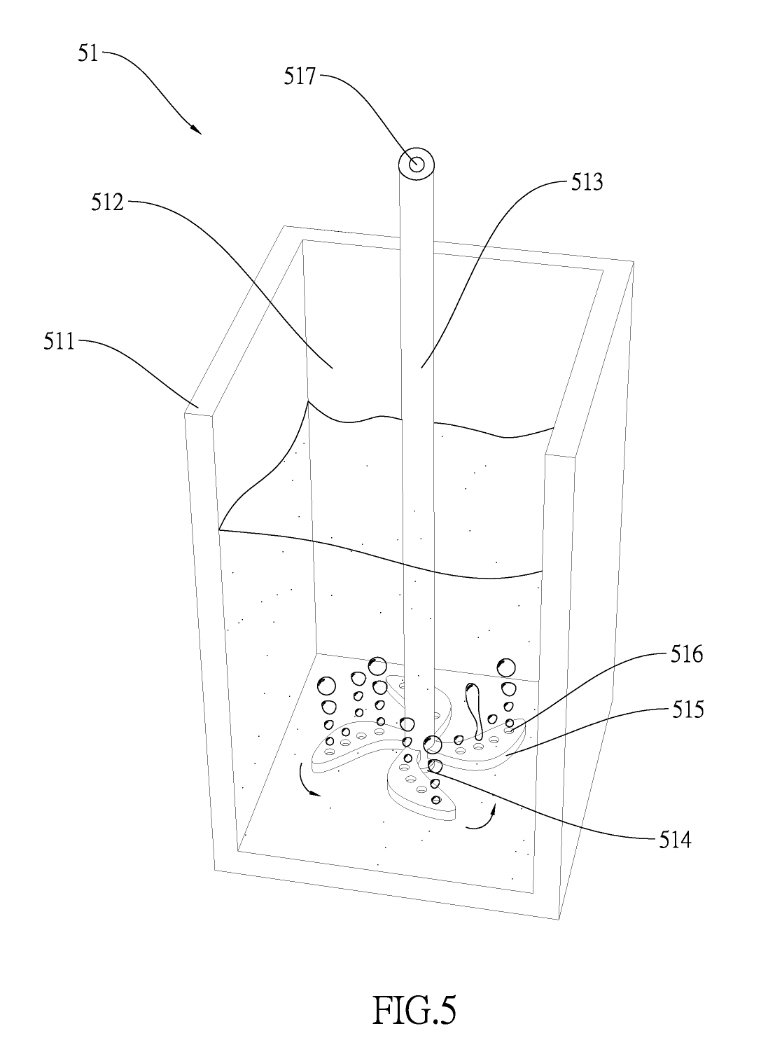

Step 7: Placing the solution in a fermenting device 51 (FIG. 5) for fermentation until beverage is produced wherein fermentation temperature is between 26° C. and 35° C., and fermentation time lasts for 3 to 10 days.

Step 8: Packaging the beverage in containers.

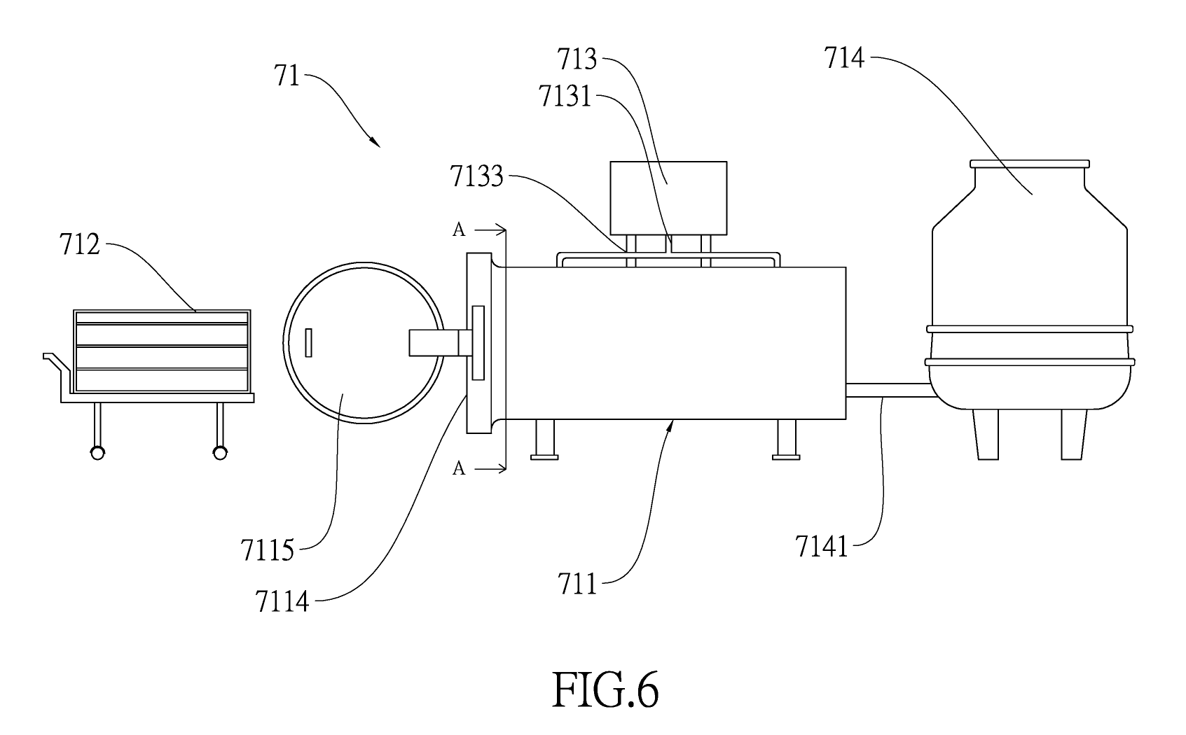

Step 9: Sterilizing the containers in a sterilizing device 71 (FIG. 6) to obtain sterilized beverage containers.

Referring to FIGS. 2 to 7, an apparatus of producing beverage by fermenting cactus fruit and pitaya in accordance with the invention is shown and comprises the following components as detailed below.

As shown in FIG. 2 specifically, a drying device 11 of the apparatus comprises a rotational member 12 and a fan 13 above the rotational member 12. The clean cactus fruit and pitaya are placed on the activated rotational member 12. The fan 13 activates to blow the clean cactus fruit and pitaya for drying.

As shown in FIG. 3 specifically, a juicing device 21 of the apparatus comprises a organic member 211, an inlet 212 above the organic member 211, a driving member 214 for activating the inlet 212, an outlet 217, a rotational shaft 215 operatively connected to the driving member 214, a reciprocating screw cutter 216 formed on the rotational shaft 215, and a cutting member 213 in the inlet 212 in which a portion the inlet 212 adjacent to the reciprocating screw cutter 216 has a diameter less than that of most fruits. In operation, cactus fruit or pitaya is thrown into the inlet 212. Next, the cutting member 213 cuts cactus fruit or pitaya into small pieces. Next, the driving member 214 activates to rotate the rotational shaft 215 and thus the reciprocating screw cutter 216 so as to further cut cactus fruit or pitaya pieces into smaller pieces. Finally, juice extracted from even smaller cactus fruit or pitaya pieces flows to the outlet 217.

As shown in FIG. 4 specifically, a filtering device 31 of the apparatus comprises an internal space 312, a centrifugal drum 311 in an upper portion of the space 312, an inlet 314 above the centrifugal drum 311, a blade 315 on an edge of the centrifugal drum 311 and under the inlet 314, a mesh filter 313 on an inner surface of the centrifugal drum 311 and having a portion in contact with the blade 315, and an outlet 316 under the centrifugal drum 311 and adjacent to a bottom of the shell of the filtering device 31. In operation, juice extracted from even smaller cactus fruit or pitaya pieces flows through the inlet 314 into the centrifugal drum 311. Large particles in the juice will be filtered out by the mesh filter 313 when the centrifugal drum 311 rotates in high speed. At the same time, pure juice falls to the bottom of the space 312. The accumulated juice will flow out of the filtering device 31 via the outlet 316 after rising to a level equal to the height of the outlet 316. The blade 315 may rub the large particles accumulated on the mesh filter 313 and discharge it out of the filtering device 31.

As shown in FIG. 5 specifically, a fermenting device 51 of the apparatus comprises a tank 511, an internal space 512 in the tank 511, an agitation member 513 in the space 512 and including a driving member 514 at a bottom of the agitation member 513, a plurality of blades 515 equally spaced around the driving member 514, a plurality of holes 516 through the blades 515, and a channel 517 through the agitation member 513 and communicating with the holes 516. In operation, the solution having yeast and gram-positive bacteria is poured into the tank 511. Next, the driving member 514 is activated to rotate the blades 515 which agitate the solution. At the same time, fresh air is introduced to the solution through the channel 517 and the holes 516. Finally, yeast and gram-positive bacteria in the solution react with air to ferment the solution.

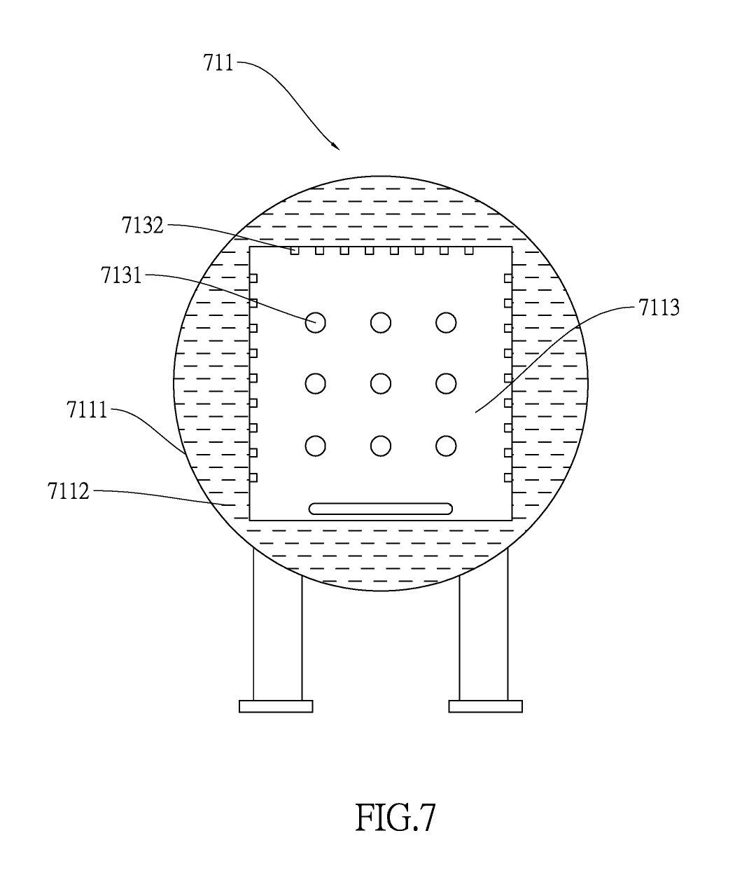

As shown in FIGS. 6 and 7 specifically, a sterilizing device 71 of the apparatus comprises a sterilization vessel 711 including a shell 7111 having an internal sterilization chamber 7113, a secondary space 7112 between the shell 7111 and the sterilization chamber 7113, an opening 7114 at an end of the shell 7111, and a gate 7115 configured to block the opening 7114; a rack 712 for holding sterilization materials configured to dispose in the sterilization vessel 711; a heating member 713 above the sterilization vessel 711 and including a plurality of first ducts 7133 for supplying hot water to the sterilization vessel 711, a plurality of sprinklers 7132 on an inner surface of the sterilization chamber 7113, and a plurality of second ducts 7131 for supplying hot water to the sprinklers 7132; and a cooling tower 714 disposed externally of the sterilization vessel 711 and including a cold water pipe 7141 communicating with the sterilization chamber 7113. In operation, half-finished products are placed on the rack 712. Then the rack 712 is placed in the sterilization vessel 711. Then the gate 7115 is closed. Next, hot water heated by the heating member 713 flows to the secondary space 7112 via the first ducts 7133 and to the sprinklers 7132 via the second ducts 7131 respectively. Next, the sprinklers 7132 sprinkle hot water on the half-finished products for sterilization purpose. It is envisaged by the invention that the purpose of sterilizing the half-finished products can be obtained due to the provisions of the sprinklers 7132 and the second ducts 7131. After the sterilizing step, warm water is drained out of the sterilization vessel 711. Further, cold water is pumped into the secondary space 7112 via the cold water pipe 7141. The cold water may lower temperature of the shell 7111 to room temperature. Finally, the products are removed out of the sterilization vessel 711.

It is envisaged by the invention that the sugar free fermentation saves time and the beverage has a lower calorie.

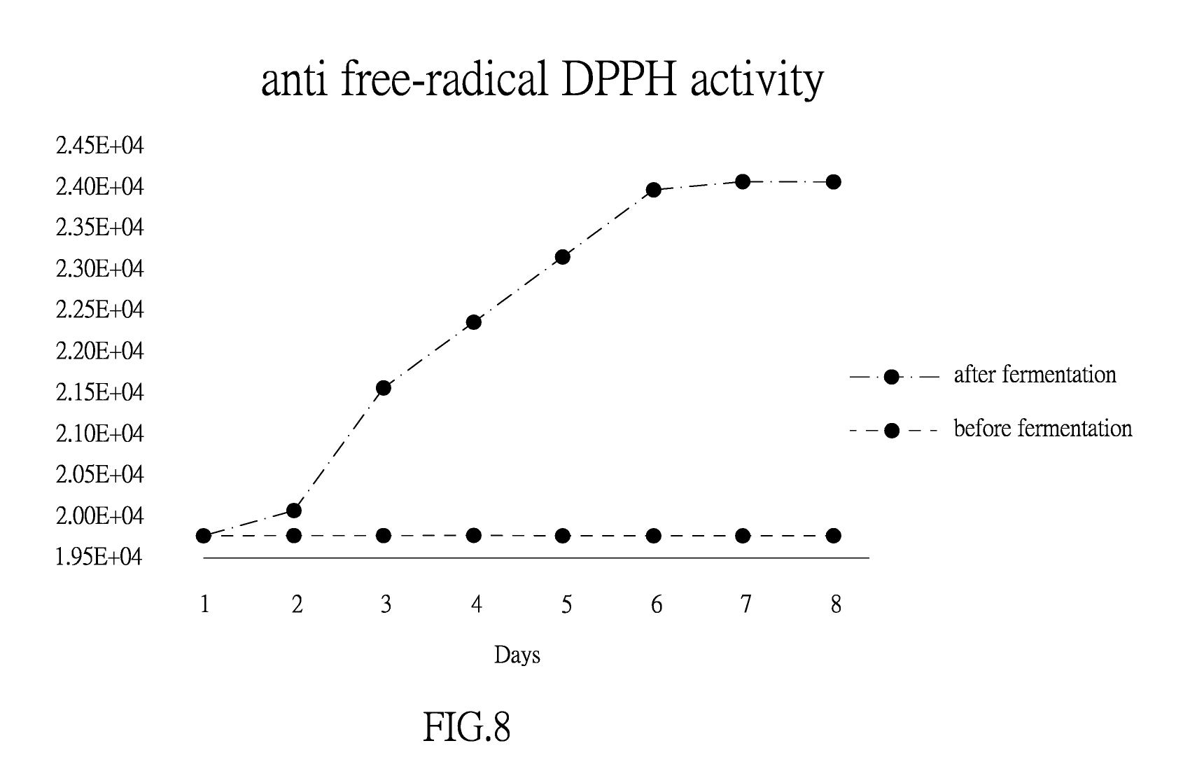

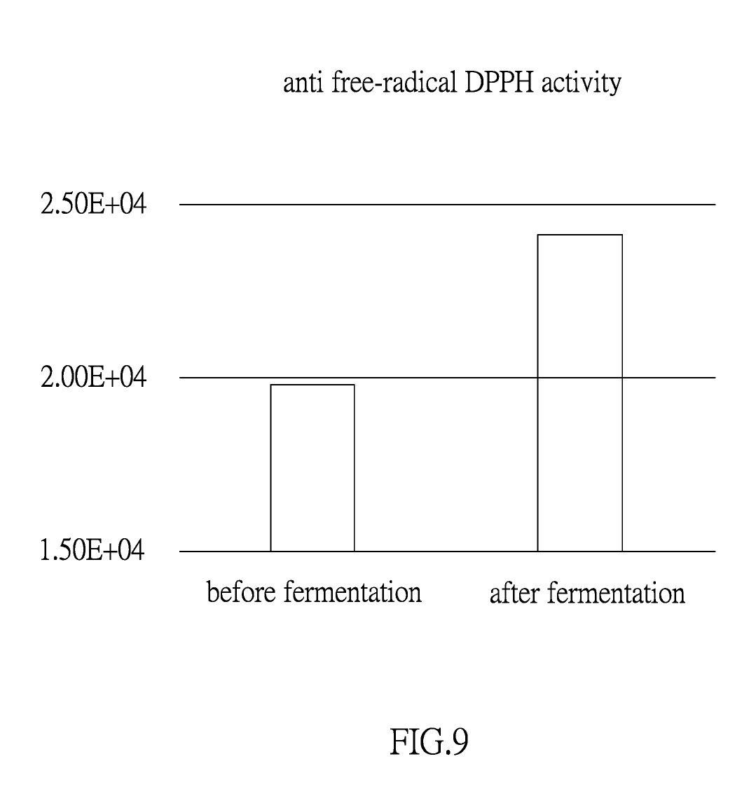

Referring to FIGS. 8 and 9, advantages of the process of the invention are shown in which FIG. 8 is a chart of days versus values for anti free-radical DPPH (2,2-diphenyl-1-picrylhydrazyl) activity before and after fermentation, and FIG. 9 is a bar chart of days versus values for anti free-radical DPPH activity before and after fermentation. Specifically, the solution before fermentation (called the solution) is compared with the beverage produced by fermentation (called the beverage) in a daily basis.

As shown in FIG. 8 specifically, in day 1, there is no difference between the solution and the beverage in terms of anti free-radical DPPH activity; in day 2, the anti free-radical DPPH activity of the beverage is greater than that of the solution; in each of days 3, 4, 5 and 6, the anti free-radical DPPH activity of the beverage is significantly greater than that of the solution as the day proceeds; and in days 7 and 8, the anti free-radical DPPH activity of the beverage is substantially no change in comparison with that of the beverage observed in day 6. It is concluded that the anti free-radical DPPH activity of the beverage is significantly greater than that of the solution.

As shown in FIG. 9 specifically, the anti free-radical DPPH activity of the beverage is about two times of that of the solution.

The invention has the following characteristics: The beverage is antioxidant. Saccharomyces boulardii, Lactobacillus acidophilus, and Bifidobacterium longum do not exclude one another and may greatly increase anti free-radical DPPH activity of the production of the beverage. The beverage is full of nutrients. Fermentation time is greatly decreased. No sugar is added in the process. Calorie of the beverage is greatly lowered.

While the invention has been described in terms of preferred embodiments, those skilled in the art will recognize that the invention can be practiced with modifications within the spirit and scope of the appended claims.

What is claimed is:

1. A process of producing beverage being antioxidant, comprising the steps of:
(1) washing cactus fruit and pitaya with clean water;
(2) drying the clean cactus fruit and pitaya;
(3) juicing the cactus fruit to obtain juice of the cactus fruit in a juicing device and juicing the pitaya to obtain juice of the pitaya in the juicing device respectively;
(4) filtering the juice of the cactus fruit and the juice of the pitaya respectively in a filtering device;
(5) mixing the filtered juice of the cactus fruit and the filtered juice of the pitaya with water to obtain a solution;
(6) pouring yeast and gram-positive bacteria in the solution wherein the yeast is Saccharomyces boulardii and the gram-positive bacteria includes Lactobacillus acidophilus and Bifidobacterium longum;
(7) fermenting the solution in a fermenting device until beverage is produced wherein fermentation temperature is between 26° C. and 35° C. and fermentation time lasts for 3 to 10 days;
(8) packaging the beverage in containers; and
(9) sterilizing the containers in a sterilizing device to obtain sterilized beverage containers;
wherein the process is characterized in that:
the juicing device comprises a organic member, an inlet above the organic member, a driving member for activating the inlet, an outlet, a rotational shaft operatively connected to the driving member, and a reciprocating screw cutter formed on the rotational shaft wherein a portion the inlet adjacent to the reciprocating screw cutter has a diameter less than that of most fruits;
the filtering device comprises an internal space, a centrifugal drum in an upper portion of the space, an inlet above the centrifugal drum, a blade on an edge of the centrifugal drum and under the inlet, a mesh filter on an inner surface of the centrifugal drum and having a portion in contact with the blade, and an outlet under the centrifugal drum and adjacent to a bottom of the shell of the filtering device;
the fermenting device comprises a tank, an internal space in the tank, an agitation member in the space and including a driving member at a bottom of the agitation member, a plurality of blades equally spaced around the driving member, a plurality of holes through the blades, and a channel through the agitation member and communicating with the holes; and
the sterilizing device comprises a sterilization vessel including a shell having an internal sterilization chamber, a secondary space between the shell and the sterilization chamber, an opening at an end of the shell, and a gate configured to block the opening, a rack for holding sterilization materials configured to dispose in the sterilization vessel; a heating member above the sterilization vessel and including a plurality of first ducts for supplying hot water to the sterilization vessel, a plurality of sprinklers on an inner surface of the sterilization chamber, and a plurality of second ducts for supplying hot water to the sprinklers; and a cooling tower disposed externally of the sterilization vessel and including a cold water pipe communicating with the sterilization chamber.

2. The process of producing beverage of claim 1, wherein the cactus fruit is the ripened ovaries of cactus and the peel of cactus.

3. The process of producing beverage of claim 1, wherein the pitaya is the ripened ovaries thereof and the peel thereof.

4. The process of producing beverage of claim 1, wherein a volumetric ratio of the filtered juice of the cactus fruit, the filtered juice of the pitaya, and water is 0.05-0.1:1-5:1-5.

5. The process of producing beverage of claim 1, wherein a volumetric ratio of Saccharomyces boulardii, Lactobacillus acidophilus, and Bifidobacterium longum is 1-5:1-3:1-3.

6. The process of producing beverage of claim 1, wherein a volumetric ratio of the solution and both yeast and gram-positive bacteria is 97-99:1-3.

7. The process of producing beverage of claim 1, further comprising a cutting member in the inlet wherein in operation, cactus fruit or pitaya is thrown into the inlet, and the cutting member cuts cactus fruit or pitaya into small pieces which are sent to the reciprocating screw cutter to further cut into smaller pieces.
